(19) United States
(12) Patent Application Publication
(10) Pub. No.: US 2014/0239088 A1
(43) Pub. Date: Aug. 28, 2014

(54) HANGING SCENTED BEAD AIR FRESHENER

(71) Applicant: BATH SOLUTIONS, INC., Elk Grove Village, IL (US)

(72) Inventor: Thomas H. Wolf, Elk Grove Village, IL (US)

(73) Assignee: Bath Solutions, Inc, Elk Grove Village, IL (US)

(21) Appl. No.: 13/780,062

(22) Filed: Feb. 28, 2013

(51) Int. Cl. A61L 9/12

(52) U.S. Cl. CPC A61L 9/12
USPC 239/58

(57) ABSTRACT

This concerns a hangable air freshener that houses aroma-emanating material, such as a plurality of scented beads. The hangable air freshener has a cover bearing a hook, which cover is movable from a fully closed condition to an open condition. When at the opened condition, access is provided to a seal which when compromised allows for passage of fragrance from the scented material out of the air freshener and into the surroundings. The cover is replaceable and allows for fragrance passage as desired.

### TECHNICAL FIELD

The present subject matter relates to air fresheners with a source of fragrance in the form of beads or other non-liquid fragrance sources. More particularly, the present subject matter relates to hanging scented air fresheners including a volume of fragrance sources such as aroma-emanating beads and a hook from which the air freshener can be suspended for various household-type uses.

### BACKGROUND

Air fresheners are a common source of providing pleasant fragrances at locations in need of freshening. Air fresheners typically are manufactured, shipped, stored in warehouses, purchased and stored at households or the like before the ultimate user decides to place the air freshener into service. Many air fresheners are configured to rest upon a surface at the desired air freshening location. The present disclosure provides the observation that it would be advantageous to provide air fresheners that remain securely sealed to avoid premature fragrance escape and that do not require a horizontal surface as a resting place once ready for air freshening use.

### SUMMARY

There are several aspects of the present subject matter that may be embodied separately or together in the devices described and claimed below. These aspects may be employed alone or in combination with other aspects of the subject matter described herein, and the description of these aspects together is not intended to preclude these aspects separately or the claiming of such aspects separately or in different combinations as may be set forth in the claims appended hereto.

In one aspect, an air freshener has a contained volume within which non-liquid fragrance sources such as scented aroma emanating beads are positioned and that has a hook by which a user places the device so as to be suspended from a location suitable for hanging.

According to another aspect, a hangable air freshener of fragrance sources such as aroma-emanating scented beads, has a slidable cover that overlies a breakable seal, the cover being slidable to expose the seal to unsealing action, the cover having a hook from which the device can be suspended at a location at which air freshening is desired.

In yet another aspect, an air freshener device containing non-liquid fragrance sources such as scented beads has a cover surface that is oriented at a non-perpendicular angle with respect to sidewalls of the container such that the cover is not horizontal when in use and has a hook component of the cover at a location elevated with respect to other portions of the container.

In a further aspect, an air freshener contains a volume of scented beads or other non-liquid fragrance source within a container with an opening having a slidable cover and a hook projecting from the cover, whereby the cover provides access to the container and a means for suspending the air freshener without having the air freshener rest upon a horizontal surface.

In a further aspect, the device has a body containing a plurality of scented beads or the like, the device having a slidable cover that protects a frangible seal thereunder and that allows sliding access to the seal so that same may be compromised or substantially removed to allow for escape of the fragrance from the scented beads through apertures in the cover that is then used to close the container to prevent escape of the scented beads themselves.

In yet a further aspect, a hangable air freshener is provided with a container having sidewalls, a bottom wall, a top wall and an enclosed volume generally therewithin containing a plurality of aroma-emanating beads or the like, the top wall having an access opening through which the beads or the like enter and can be replaced in select embodiments, the top wall having a support surface. A seal of a sheet or film has a first condition overlying and sealing the access opening and a second condition at which the access opening is unsealed. In addition, a cover is slidable over the support surface and has a first position at which the cover overlies and interferes with access to the seal and a second position at which the cover exposes and allows access to and breaching of the seal, the cover being perforate such that aroma from the aroma-emanating beads or the like is allowed to pass out of the container and into the environment within which the air freshener is positioned once the seal is broken and the cover is repositioned on the air freshener. The cover further includes a hook projecting away from the support surface and that is sized and shaped to impart hanging capability to the air freshener.

In yet a further aspect, a hangable air freshener is provided with a container having sidewalls, a bottom wall, a top wall and an enclosed volume generally therewithin containing a plurality of aroma-emanating beads or the like, the top wall having an access opening through which the aroma-emanating members enter and can be removed for replacement in select embodiments, the top wall having a support surface. A seal of a sheet or film has a first condition overlying and sealing the access opening and a second condition at which the access opening is unsealed. In addition, a cover is slidable over the support surface and has a first position at which the cover overlies and interferes with access to the seal and a second position at which the cover exposes and allows access to and breaching of the seal, the cover being perforate such that aroma from the aroma-emanating members is allowed to pass out of the container and into the environment within which the air freshener is positioned once the seal is broken and the cover is repositioned on the air freshener. The cover further includes a hook projecting away from the support surface and being sized and shaped to impart hanging capability to the air freshener. The cover is non-parallel to the bottom wall of the container, typically being planar and oriented at an acute angle with respect to the bottom wall.

In yet a further aspect, a hangable air freshener is provided with a container having sidewalls, a bottom wall, a top wall and an enclosed volume generally therewithin containing a plurality of aroma-emanating members, the top wall having an access opening through which the aroma-emanating members enter and can be replaced in select embodiments, the top wall having a support surface. A seal of a sheet or film has a first condition overlying and sealing the access opening and a second condition at which the access opening is unsealed. In addition, a cover is slidable over the support surface and has a first position at which the cover overlies and interferes with access to the seal and a second position at which the cover exposes and allows access to and breaching of the seal, the cover being perforate such that aroma from the aroma-emanating members is allowed to pass out of the container and into the environment within which the air freshener is positioned once the seal is broken and the cover is repositioned on the air freshener. The cover further includes a hook projecting away from the support surface and being sized and shaped to impart hanging capability to the air freshener. The hook is unitary with the cover and positioned at a location of the cover closer to the first end than to the second end of the cover.

In another aspect, a hangable air freshener is provided with a container having sidewalls, a bottom wall, a top area and an enclosed volume generally therewithin, the top area having a first end portion and a second end portion, a plurality of aroma-emanating beads being within the enclosed volume. The top area has an access opening sized to permit passage therethrough of the aroma-emanating beads. Also provided is a seal sheet having a first condition overlying and sealing the access opening, the seal sheet having a second condition at which the access opening is unsealed, as well as a cover of the top area, the cover having a closed position at which the cover overlies and interferes with access to the seal sheet and to the access opening, the cover having an open position at which the cover at least partially exposes and allows access to and breaching of the seal sheet thereby unsealing the access opening. This cover is perforate such that, in the absence of the access opening being sealed but while the cover overlies the access opening, aroma from the aroma-emanating beads is allowed to pass out of the container and into the environment within which the air freshener is positioned. A hook component of the cover projects away from the support surface of the top area of the container, the hook component being sized and shaped to impart hanging capability to the air freshener. The sidewalls include opposing sidewalls, one sidewall being at an acute angle with the first end portion of the top area, its opposing sidewall being at an obtuse angle with the second end portion of the top area, and the hook component is at a location of the cover closer to said first end portion than to said second end portion of the top area.

### BRIEF DESCRIPTION OF THE DRAWINGS

FIG. 1 is a perspective view of a hangable air freshener in accordance with one embodiment, including scented beads therewithin;

FIG. 2 is a side elevational view of the embodiment of FIG. 1, same including a wider sidewall;

FIG. 3 is a side elevational view of the embodiment of FIG. 1, same including a narrower sidewall;

FIG. 4 is a partial longitudinal cross-sectional view through the upper portion of FIG. 2;

FIG. 5 is a partial transverse cross-sectional view through the upper portion of FIG. 2;

FIG. 6 is a partial longitudinal cross-sectional view similar to FIG. 4 showing sliding movement of the cover for exposing the seal and/or removing or adding non-liquid aroma-emanating members such as scented beads;

FIG. 7 is a partial transverse cross-sectional view similar to FIG. 5, with the cover fully removed and the seal in place and intact;

FIG. 8 is a partial longitudinal cross-sectional view similar to FIG. 6, with the cover completely removed; and

FIG. 9 is a partial transverse cross-sectional view similar to FIG. 5 after removal of the seal and re-positioning of the cover so as to contain the fragrance beads or the like while allowing fragrance escape from the air freshener.

### DETAILED DESCRIPTION OF THE ILLUSTRATED EMBODIMENTS

The embodiments disclosed herein are exemplary only, and the subject matter described herein may be embodied in various forms. Therefore, specific details disclosed herein are not to be interpreted as limiting the scope of the subject matter as defined in the accompanying claims.

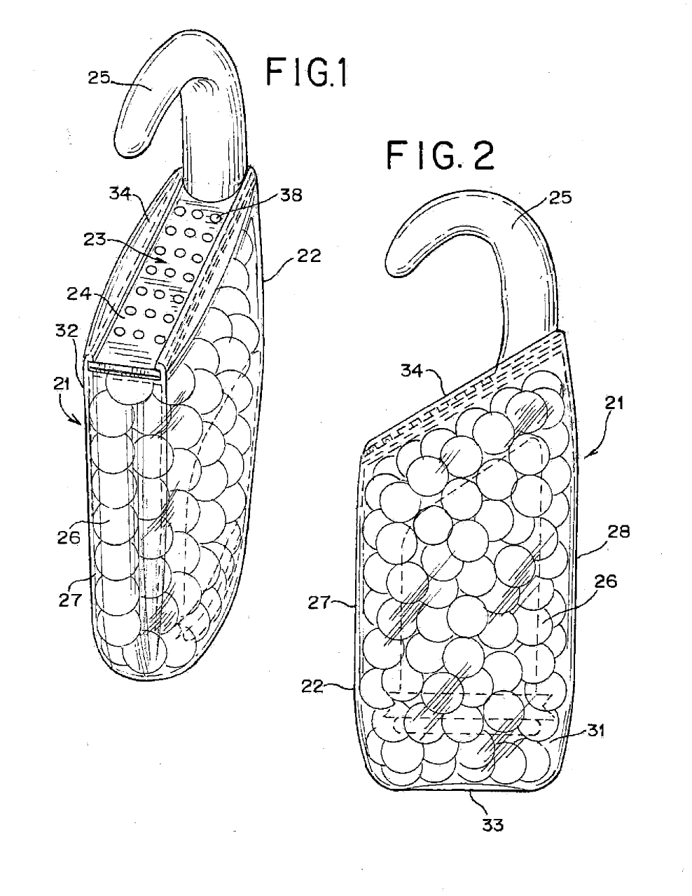

An embodiment of a hangable air freshener 21 is depicted in FIG. 1 and FIG. 2. This includes a container 22 and a cover generally designated at 23. The cover includes a cover panel 24 and a hook component 25 projecting upwardly (as seen in the drawings) from the cover panel 24.

A plurality of non-liquid fragrance sources 26 are positioned within the container 22, typically substantially filling the container. When used herein, the term “scented beads” is used at various times to encompass non-liquid fragrance sources whether or not spherical in shape. Scented beads 26 are of generally known construction, composition and properties, being impregnated with fragrance material that releases from the bead when the bead is exposed to air or the environment. These scented beads are known to be available in a variety of aromas and colors. Various specific properties of these scented beads are known to one of ordinary skill in the art. They may or may not be substantially spherical.

In some embodiments, it will be observed that at least one wall of the container 22 is non-opaque, such as being translucent or transparent, whereby the presence of scented beads within the container can be understood to be present therewithin. In one embodiment, all of the sidewalls can be transparent, as generally shown in FIG. 1. The sidewalls include opposing narrower sidewalls 27 and 28, and opposing wider sidewalls 31 and 32. Any one of sidewalls 27, 28, 31, 32 or a plurality of them can be non-opaque depending upon the extent that one wishes to have the scented beads generally visible within the container 22. Also possibly non-opaque can be the bottom wall 33 and/or the top wall 34. Any of these non-opaque components may be transparent, translucent or other condition that permits features of the scented beads to be discernible from outside of the closed container.

The hook component is illustrated in the drawings as having a configuration typical of a hook for hanging a device within a household context. As illustrated, the hook component 25 that is shown in the drawings receives the support from which it is to be hung at a location above the container 22, the curve of the hook component being over the container as opposed to any direction oriented away from the container. The particular shape of the hook component is variable; for example, rather than being generally curved as shown in the drawings, the hook can have walls or surfaces that are generally flat and meet at angles with other walls or surfaces. Generally, the hook component will have a curved configuration where the inside surface of the hook component tends to be more compatible with a typical household member from which the hangable air freshener can be hung, such as a dowel or rod within a closet or the like.

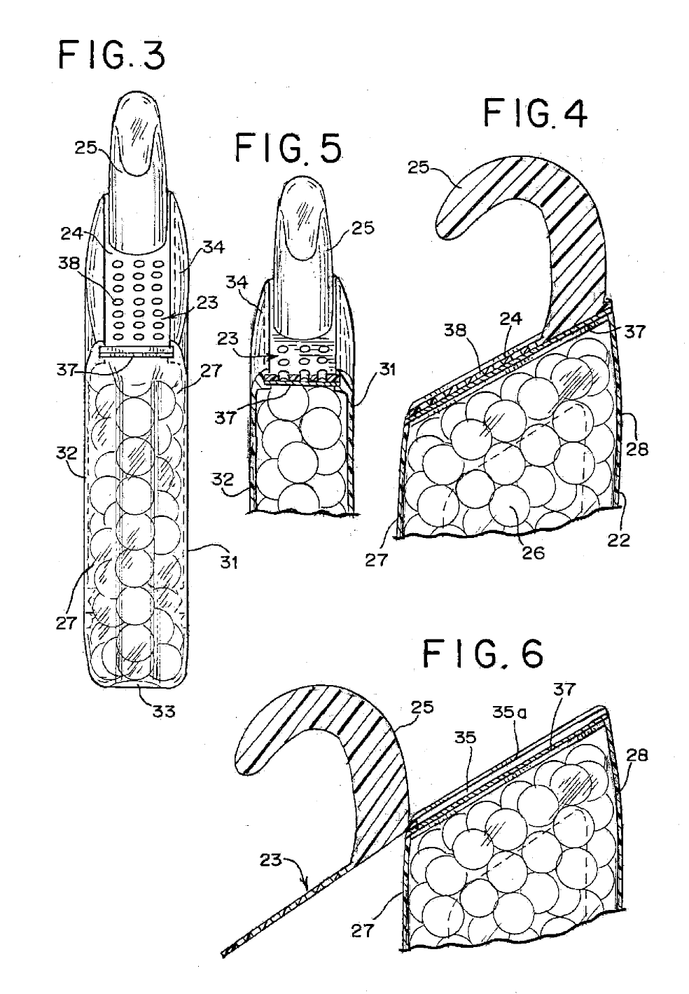

An important feature of the cover is that same is movable from a first position at which the cover closes the container (as in FIGS. 1-5 and 9) to a second position (as in FIGS. 6-8) at which the cover allows access to portions of the air freshener scented beads that are otherwise under the cover when it is closed. In an illustrated embodiment, this function is achieved by a sliding action to change between the cover first position at which the cover overlies and interferes with access to the container and the cover second position at which the cover at least partially exposes and allows access to the container areas that had been underlying the cover.

As illustrated in this embodiment, the cover includes a flat or planar cover panel 24. This cover panel 24 is sized and shaped to slidably engage a complementary member such as the illustrated channel or rib 35 that provides a support surface for the cover panel 24. The channel 35 can be associated with or replaced by other structures such as a rib or ribs which can be positioned along the lines of that shown for the channel or channels. Opposing channel or rib 35a can be included in order to provide support function along opposing edges of the cover panel 24. It will be appreciated that channel 35 is shown in the top wall of the container. When desired, the top wall 34 of the container can be considered an extension of the opposing wider sidewalls 31, 32 in a generally curved fashion. There is no requirement that the top wall be itself substantially flat. It merely is to be of a shape that will accommodate the movability of the cover 23 while providing structure that allows the container to be fully closed when desired and openable as discussed herein.

In the illustrated embodiment, the top wall 34 includes an access opening 36. This access opening can be used to fill the container with the scented beads 26 either at the factory or by the consumer when a refillable hangable air freshener is desired. When the access opening is to fulfill this function, the access area provided thereby must be large enough to permit passage of the scented beads therethrough.

When the scented beads are passed through the access opening 36 during manufacture of the hangable air freshener 21, the access opening is to be sealed so that the fragrance of the scented beads 26 will remain within the container during warehousing, transporting, retailing and storage in the household before use. A suitable manner of achieving this objective is to include a seal sheet or film 37, such as shown in FIGS. 4-7. Seal sheet 37 can take the form of a thin film that provides an airtight seal such as found in over-the-counter pharmaceutical compositions, certain air freshener packaging, food products and the like. These can be of a so-called foil type and/or be of multiple layers or laminates having barrier capabilities in one or more layers. Barrier layers can be metallic, polymeric or combinations thereof.

The seal sheet 37 is affixed to surfacing along the top wall, for example, around the access opening, by adhesive means, heat sealing, sonic welding, or other means of attachment known in the art. When thus attached, the seal sheet 37 secures the scented beads within the compartment and provides barrier functions noted herein. In addition, the seal sheet is frangible so that the seal can be compromised and/or removed partially or completely in order to reduce or remove barriers to fragrance dissemination out of the container containing the scented beads. It is generally preferred that this frangibility feature be such that the seal sheet is removable, penetrable or otherwise compromised without requiring specially designed tools, usually requiring something of only moderate sharpness such as a pointed object, a fingernail or by concerted digital force imparted to the seal sheet by the user.

Cover panel 24 is perforate; for example, same may include one or more apertures 38 completely through the cover panel. In the embodiment illustrated, the cover panel 24, as well as the support surface and top wall, are oriented at an angle with respect to the container body. More specifically, as can be seen for example in FIG. 4, the cover panel 24 meets one of the opposing narrower sidewalls 28 at an acute angle and meets the other opposing sidewall 27 at an obtuse angle (both angles being measured from the inside of the container), thereby created the slanted or angled top portion of the container that is illustrated. In the embodiment illustrated in FIG. 2, it will be appreciated that the access opening 36 or top wall 34 is non-parallel with respect to the bottom wall 33. Stated otherwise, the top wall is spaced farther from the bottom wall 33 for a distance that is longer along opposing narrow sidewall 28 than along opposing narrow sidewall 27.

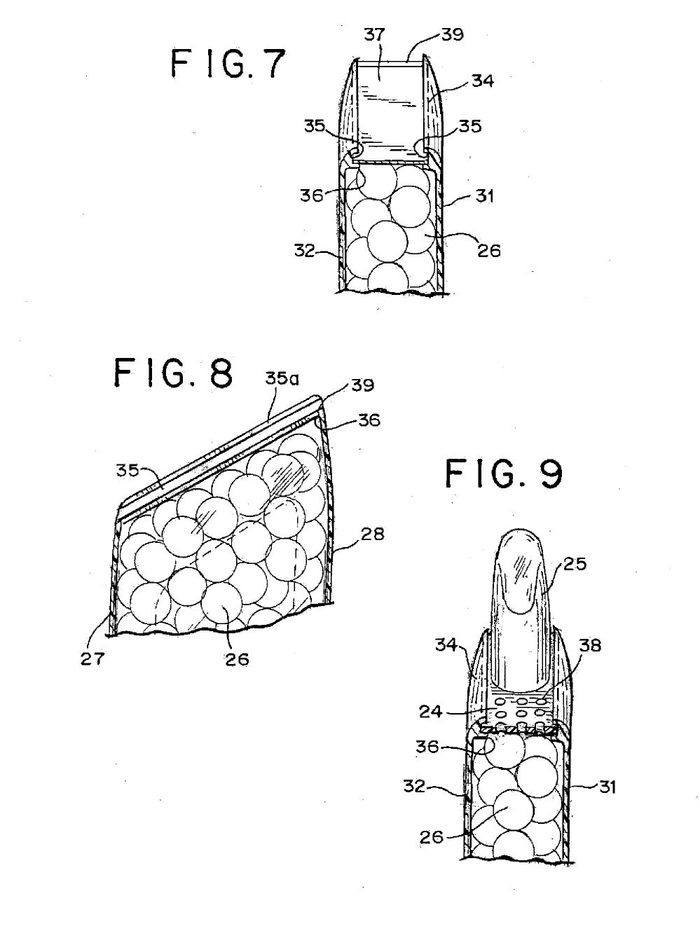

Also associated with the cover in the illustrated embodiment is a structure to provide a stop function to sliding movement of the cover 23. This function can be provided by a stop 39 (FIG. 7 and FIG. 8) that prevents the cover 23 from moving along the container in an upward or outward direction at which same would otherwise be disposed to move when the air freshener 21 is suspended by its hook component 25. Instead, stop 39 interferes with sliding movement of the cover panel 24 within the channel 35 beyond and to the right as in FIG. 4, for example, of the container 22. It will be seen from FIG. 6 that the cover panel 24 and hence the cover 23 and hook component 25 are able to move outwardly and downwardly generally to the left as oriented in FIG. 6. In the illustrated embodiment, the cover can be completely removed from the container.

In use, appropriate measures are taken to move the cover 23 to a position as generally shown in FIG. 6, thereby allowing the consumer to gain access to the seal sheet 37 which is otherwise overlain by the cover 23, thereby blocking access to the seal sheet, as seen in FIGS. 1-5. In the illustrated embodiment, the user of the hangable air freshener can totally remove the cover 23 from the rest of the hangable air freshener 21 or can effect only a partial removal if desired. With the seal sheet or film is thus exposed at least partially, the user compromises the seal by either removing the seal sheet (or a majority of the seal sheet), which can be facilitated by the use of a peel tab or the like of the type generally known in the art. Alternatively, the user can fracture the seal sheet or film by a simple sharp object, by a fingernail, or by the user of digital force in order to create an opening or fracture in the seal, which in many instances is adequate to allow sufficient passage of fragrance from the scented beads and into the atmosphere surrounding the hangable air freshener.

Once the seal has been compromised to whatever extent desired by the user, the cover 23 is repositioned on top of the container 22. This is illustrated particularly in FIG. 8 and FIG. 9 in the situation where the seal sheet is substantially completely removed from the access opening 36 and the cover is slid back in place. With this configuration, fragrance from the scented beads 26 is able to escape from the container by way of aperture or apertures 38. At the same time, the hangable air freshener is in a condition for hanging, such as over a bar or rod of a closet or any other location in need of air freshening as provided by fragrance from a passive source, the illustrated passive source being scented beads 26 and fragrance escape through the aperture or apertures 38.

With the illustrated embodiments, the hangable air freshener can be of a refillable variety. More specifically, with the seal sheet or film removed or otherwise substantially compromised, the user can remove spent scented beads out of the container through the access opening Thereafter, fresh scented beads can then be inserted into the container through the access opening. This is followed by return of the cover to its first condition overlying and sealing the access opening, at which time the hangable air freshener can be repositioned where desired within a household or other location for air freshening purposes.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features thereof, including combinations of features that are individually disclosed or claimed herein.

1. A hangable air freshener, comprising:
a container having sidewalls, a bottom wall, a top area and an enclosed volume generally therewithin;
a plurality of aroma-emanating beads within the enclosed volume;
said top area has an access opening sized and shaped to permit passage therethrough of aroma-emanating beads, the top area having a support surface along the access opening;
a seal sheet having a first condition overlying and sealing the access opening, the seal sheet having a second condition at which the access opening is unsealed;
a cover slidable over the support surface, the cover having a first position at which the cover overlies and interferes with access to the seal sheet and closes the access opening, the cover having a second position at which the cover at least partially exposes and allows access to and breaching of the seal sheet thereby unsealing the access opening;
said cover being perforate such that, in the absence of the access opening being sealed, aroma from the aroma-emanating beads is allowed to pass through the perforate cover when same overlies the access opening and out of the container and into the environment within which the air freshener is positioned; and
a hook component of the cover that projects away from the support surface of the top area of the container, the hook component being sized and shaped to impart hanging capability to the air freshener.

2. The hangable air freshener of claim 1, wherein said cover is slidable along the support surface until the cover is fully removed from the support surface.

3. The hangable air freshener of claim 2, wherein when the cover is fully removed, the cover no longer overlies the access opening

4. The hangable air freshener of claim 3, wherein the access opening is fully open when the cover is removed and the seal sheet is breached.

5. The hangable air freshener of claim 1, wherein said sidewalls include two first opposing sidewalls and two second opposing sidewalls narrower than the first opposing sidewalls, and the support surface is planar, is at an acute angle with one of said second opposing sidewalls, and is at an obtuse angle with another of said second opposing sidewalls.

6. The hangable air freshener of claim 1, wherein said sidewalls include two first opposing sidewalls and two second opposing sidewalls narrower than the first opposing sidewalls, and the top area of the container is planar, is at an acute angle with one of said second opposing sidewalls, and is at an obtuse angle with another of said second opposing sidewalls.

7. The hangable air freshener of claim 1, wherein said sidewalls include two first opposing sidewalls and two second opposing sidewalls narrower than the first opposing sidewalls, the support surface has two ends, a first such end being at an acute angle with one of said second opposing sidewalls, a second such end being at an obtuse angle with another of said second opposing sidewalls, and the hook component is at a location of the cover closer to said first end than to said second end of the support surface.

8. The hangable air freshener of claim 7, further including a stop at which the cover and the top wall engage to prevent sliding movement beyond the second opposing sidewall with respect to which the first end is at an acute angle.

9. The hangable air freshener of claim 1, wherein the support surface of the top area includes a rib along which the cover is slidable.

10. The hangable air freshener of claim 1, wherein the top area includes a channel within which the cover is slidable.

11. The hangable air freshener of claim 1, wherein at least one said wall of the container is non-opaque thereby allowing observation of at least a portion of at least some of the aroma-emanating beads with an unaided eye.

12. A hangable air freshener, comprising:
a container having sidewalls, a bottom wall, a top area and an enclosed volume generally therewithin, the top area having a first end portion and a second end portion;
a plurality of aroma-emanating beads within the enclosed volume;
said top area has an access opening sized and shaped to permit passage of the aroma-emanating beads therethrough;
a seal sheet having a first condition overlying and sealing the access opening, the seal sheet having a second condition at which the access opening is unsealed;
a cover slidable along the top area, the cover having a first position at which the cover overlies and interferes with access to the seal sheet and closes the access opening, the cover having a second position at which the cover at least partially exposes and allows access to and breaching of the seal sheet thereby unsealing the access opening, the cover having a first end and a second end;
said cover being perforate such that in the absence of the access opening being unsealed aroma from the aroma-emanating beads is allowed to pass through the perforate cover and out of the container and into the environment within which the air freshener is positioned;
a hook component of the cover that projects away from the top area of the container, the hook component being sized and shaped to impart hanging capability to the air freshener;
said sidewalls include two first opposing sidewalls and two second opposing sidewalls narrower than the first opposing sidewalls, and the top area is non-parallel to said bottom wall; and
the hook component is unitary with the cover and positioned at a location of the cover closer to said first end than to said second end of the cover.

13. The hangable air freshener of claim 12, further including a stop at which the cover and the top area engage to prevent sliding movement beyond the second opposing sidewall with respect to which the first end is at an acute angle.

14. The hangable air freshener of claim 13, wherein the top area includes a channel within which the cover is slidable.

15. The hangable air freshener of claim 12, wherein he seal sheet is a flexible film.

16. A hangable air freshener, comprising:
a container having sidewalls, a bottom wall, a top area and an enclosed volume generally therewithin, the top area having a first end portion and a second end portion;
a plurality of aroma-emanating beads within the enclosed volume;
said top area has an access opening sized to permit passage therethrough of the aroma-emanating beads;
a seal sheet having a first condition overlying and sealing the access opening, the seal sheet having a second condition at which the access opening is unsealed;
a cover of the top area, the cover having a closed position at which the cover overlies and interferes with access to the seal sheet and to the access opening, the cover having an open position at which the cover at least partially exposes and allows access to and breaching of the seal sheet thereby unsealing the access opening;
said cover being perforate such that, in the absence of the access opening being sealed but while the cover overlies the access opening, aroma from the aroma-emanating beads is allowed to pass out of the container and into the environment within which the air freshener is positioned;
a hook component of the cover that projects away from the support surface of the top area of the container, the hook component being sized and shaped to impart hanging capability to the air freshener; and
said sidewalls include opposing sidewalls, one sidewall being at an acute angle with the first end portion of the top area, its opposing sidewall being at an obtuse angle with the second end portion of the top area, and the hook component is at a location of the cover closer to said first end portion than to said second end portion of the top area.

17. The hangable air freshener of claim 16, wherein the cover has a cover panel, and the hook component and cover panel are of one-piece unitary construction.

18. The hangable air freshener of claim 16, wherein the top area is non-parallel to the bottom wall.

19. The hangable air freshener of claim 16, wherein at least one said wall of the container is non-opaque thereby allowing observation of at least a portion of at least some of the aroma-emanating beads with an unaided eye.
